(19) United States
(12) Patent Application Publication
(10) Pub. No.: US 2009/0041837 A1
(43) Pub. Date: Feb. 12, 2009

(54) Aptamers utilized as targeting agents for pharmaceutical compounds used in the treatment of cancer and other diseases

(76) Inventors: Henry John Smith, Temecula, CA (US); James Roger Smith, Laguna Niguel, CA (US)

Correspondence Address: HENRY JOHN SMITH, 40670 CALLE TORCIDA, TEMECULA, CA, 92591, US

(21) Appl. No.: 12/221,430

(22) Filed: Aug. 4, 2008

Related U.S. Application Data
(60) Provisional application No. 60/963,435, filed on Aug. 6, 2007.

(51) Int. Cl. A61K 9/127; A61K 9/14; A61P 35/00

(52) U.S. Cl. 424/450; 424/490

(57) ABSTRACT

This invention describes a method whereby selected aptamers are used as a carrier compound to deliver drug nanoparticles and/or drug liposomes to the tumor or disease site. These aptamers have the propensity to localize in areas of tissue necrosis. Many tumors have areas of necrosis that can be targeted using aptamer coated drug nanoparticles and/or aptamer coated drug liposomes. Similarly many infectious diseases and autoimmune diseases have areas of inflammation and/or areas of necrosis and can also be targeted using aptamer coated drug nanoparticles and/or aptamer coated drug liposomes.

### CROSS-REFERENCE TO RELATED APPLICATIONS

This utility patent application claims priority to Provisional Patent Application Ser. No. 60/963,435 filed Aug. 21, 2007 entitled APTAMERS UTILIZED AS TARGETING AGENTS FOR PHARMACEUTICAL COMPOUNDS USED IN THE TREATMENT OF CANCER AND OTHER DISEASES

### REFERENCES

Autoantibodies Utilized as Carrier Agents for Pharmaceutical compounds used in Tumor imaging and Cancer treatment. Smith H. J. et al. U.S. patent application Ser. No. 10/745,308 Cancer Nanotechnology. National Cancer Institute. NIH Publication No 04-5489 January 2004 Monograph.

Systematic Evolution of Ligands by Exponential Enrichment (SELEX). Gold L. et al U.S. Pat. No. 5,270,163

Aptamer-conjugated nanoparticles for the collection and detection of multiple cancer cells. Smith J. E. et al. Anal. Chem Apr. 15, 2007;79(8):3075-82

Tumor targeting by an aptamer. Hicke B. J. et al. j. Nucl. Med April 2006; 47(4):668-78

Targeted nanoparticle-aptamer bioconjugates for cancer chemotherapy in vivo Farokhzad O. C. et al Proc. Natl Acad Sci USA Apr. 18, 2006;103(16):6315-6320

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The main applications of this invention are in developing improved methods for the treatment of cancer, infectious disease and autoimmune disease. One out of every four people in the US will die from cancer. There is tremendous interest in developing improved methods of cancer therapy. Early research on targeting tumors used antibodies obtained from immunized animals. Subsequent studies have been almost exclusively devoted to developing monoclonal antibodies against tumors. Much of the research has focused on developing antitumor antibodies using monoclonal antibodies produced by murine hybridomas. There is however a problem when murine monoclonal antibodies are injected into cancer patients there is a risk that the patient may develop an immune response against the “foreign” protein making further treatment ineffective. In order to avoid this problem there is intensive research into developing methods to “humanize” the monoclonal antibodies by substituting parts of the mouse antibody with human components or by developing fully human monoclonal antibodies.

This invention describes an alternative method of targeting tumors and other diseases using specific aptamers that can bind to necrotic cellular elements found in areas of tissue necrosis within tumors and in other diseases that have tissue damage.

Aptamers are small (i.e., 40 to 100 bases), synthetic oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration as a result of the composition of the nucleotide bases in the chain causing the molecule to fold in a particular manner. Because of their folded structure each aptamer will bind selectively to a particular ligand in a manner analogous to an antibody binding to its antigen.

There is intense research to identify aptamers that have selective binding capacity for tumor associated ligands, and to use these tumor targeting aptamers either to directly inhibit the tumor and/or as a carrier to transport cancer drugs to the tumor. Current thinking is that for an aptamer to be effective it must bind directly to the surface of the tumor cell.

The novelty of the present invention is that the targeting aptamers described herein are not directed against viable cells but are instead directed against cellular components released from dead cells. The second novel aspect of this invention is that instead of searching for aptamers that binds to a ligand that is disease specific such as a particular cell marker protein on the surface of a cell. (e.g. tumor antigen or cell marker protein) this invention describes the use of aptamers that are not specific for a particular cell type but instead are reactive with intracellular constituents common to all animal cells.

It is well known that many diseases result in some degree of tissue destruction. Many tumors have areas of necrosis and these necrotic areas contain elevated levels of intracellular material released from dead or dying cells. Similarly, many diseases such as infectious diseases also have areas of necrosis within the infection site. Similarly, autoimmune and inflammatory diseases may also have areas of necrosis within the inflammation site. This invention postulates that dead cells will release their intracellular constituents into the extracellular environment. The extracellular expressed material includes nuclear materials such as the nuclear membrane, nucleoproteins, DNA, histones etc. and cytoplasmic components such as mitochondria, ribosomes and cytoplasmic proteins. The released material being expressed extracellularly can now be the target of aptamers designed to bind to various intracellular components. The aptamers of this invention have the ability to bind within these areas of necrosis found in many different diseases.

As the aptamers of this invention target material from dead cells they are unable to directly affect viable cells where the intracellular components are protected within the living cell. This invention describes the use of these aptamers to “coat” drug nanoparticles and/or drug enclosed liposomes and cause them to become localized within the tumor or at the site of disease. The drug or cytotoxic agent is released at the disease site where it will have maximum therapeutic effect. Normal healthy tissues do not have necrotic areas and will therefore not be targeted by the aptamers of this invention.

A further benefit of this invention is that these aptamers appear to have little or no toxicity to the patient. They can be used repeatedly without provoking an adverse reaction.

### SUMMARY OF THE INVENTION

This invention describes the novel use of aptamers as carrier agents for pharmaceutical compounds used to treat cancer and other diseases. The invention describes the utilization of aptamers that have the capacity of binding to intracellular components released from dead or dying cells. Further, this invention describes the process whereby these aptamers are used to coat drug nanoparticles and/or drug liposomes in order to cause the drug to localize within the necrotic areas found in many tumors, infectious diseases and inflammatory sites in autoimmune disorders.

There are two components to this invention. First, there is the preparation and composition of drug nanoparticles or drug liposomes to impart certain properties to the drug that will enhance its efficacy and safety. Second, is to “coat” the drug nanoparticle or drug liposome with the aptamers described herein in order to further improve the safety and efficacy profile of the drug by causing it to selectively localize at the disease site where it will have optimum effect.

The aptamers are non-immunogenic and non-toxic and can be used repeatedly as “carriers” for pharmaceuticals without provoking an adverse reaction in the patient.

### DESCRIPTION OF THE INVENTION

This invention describes a method for improved delivery of pharmaceutical agents to treat a variety of tumors and other diseases using specific aptamers combined with the pharmaceutical compounds in order to improve their therapeutic and/or safety characteristics. In order to improve the “solubility” and bioavailability of the drug it can be made into submicron sized particles called “nanoparticles”. Although the term nanoparticle can be applied to any material that is measured in nanometers it is generally applied to particles that are between 1 nanometer and 1000 nanometers in size. Nanoparticles can be broadly recognized as two types; those particles that have a solid structure and those that have a liquid structure. For purposes of clarity in this invention the term “nanoparticle” will refer to the solid type of particle, and because of convention the term “liposome” will be used to describe the liquid type. In general, if the drug is insoluble or poorly soluble in physiological solution it is made into nanoparticles or incorporated into the lipid membrane of liposomes. If the drug is soluble in physiological solution it is typically encapsulated into liposomes. In certain instances the liquid drug may be incorporated in a solid or gel-matrix which is then made into nanoparticles.

The drug nanoparticle and/or the drug liposome can be treated so that the surface layer will incorporate additional chemical or biological agents. This invention describes the advantages of attaching a unique aptamer to the surface of the nanoparticle or liposome to further improve its therapeutic profile. It will also cause the drug nanoparticles or drug liposomes to accumulate within necrotic areas by binding to intracellular antigens released from dead cells. We refer to the drug delivery system described herein as a “smart” delivery system because it consists of two components: a targeting component which is the carrier aptamer, and the payload component which is the drug nanoparticle and/or drug liposome that is delivered to the disease site. Hence the terms “smart nanoparticles” and “smart liposomes”.

In a further embodiment of this invention the carrier aptamer is attached to the drug nanoparticle or drug liposome thru a polyethylene glycol (PEG) linkage molecule. The PEG coating molecule helps to protect the nanoparticle or liposome from being broken down by the liver and therefore results in more of the drug being bioavailable. This is often referred to as a “stealthy” nanoparticle and/or liposome.

This invention is based on the observation that certain aptamers have a unique propensity for binding to certain intracellular material found extracellularly within necrotic areas of certain diseases but not in healthy tissues. The invention describes the process whereby various therapeutic agents in the form of nanoparticles and/or liposomes are combined with these aptamers and used in the treatment of cancer and other diseases.

A major benefit of this invention is that these aptamers are non-immunogenic and non-toxic and therefore patients can receive repeated treatment without causing an adverse reaction in the patient.

There are a variety of aptamers can be employed as targeting carriers for pharmaceuticals. These include aptamers directed against intracellular components of the cell such as nuclear material, deoxyribonucleic acid (DNA); ribonucleoprotein (RNP), Sm antigen, ENA antigen, mitochondrial material, Golgi complex; the cytoskeleton; and also other cytoplasmic proteins found in specialized cells such as melanin in melanocytes, thyroglobulin in thyroid cells and prostate specific antigen (PSA) in prostate cells.

The aptamers may be ssDNA and/or ssRNA. In order to improve bioavailability against nucleases found in vivo the oligonucleotides may be modified to avoid nuclease attack. They may for example be synthesized as L-nucleotides instead of the natural D-nucleotides and thus avoid degradation from the natural nucleases.

Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand.

In this invention the target ligand is selected from a list of intracellular elements. For example, nuclear material (including DNA, DNP, Sm, ENA and chromatin) is known to be released from dead cells. Aptamers can be synthesized to selectively bind to one or more of these targets. In another embodiment of this invention the aptamers may be targeted to the cytoplasmic elements such as mitochondria, glogi complex; or the cytoskeleton. In another embodiment of this invention the aptamers may be targeted to other cytoplasmic material such as melanin, or thyroglobulin or PSA.

The aptamer is attached to the surface of a drug nanoparticle or drug liposome and used to selectively transport the drug to necrotic areas found within tumors or tissues damaged by infection or inflammatory diseases.

There are many methods of preparing nanoparticles known to those skilled in the art. These include dry and wet milling; super critical fluid technology, spray drying, solvent precipitation and recrystallization techniques. These methods are extensively modified to suit the requirements of the particular drug being used. These methods are known to those skilled in the art and are within the scope of this invention.

Similarly, there are many methods of preparing liposomes known to those skilled in the art. These include encapsulation, partitioning, and reverse loading techniques. As before, these methods are extensively modified to suit the requirements of the particular drug being used. These methods are known to those skilled in the art and are within the scope of this invention.

There are a large variety of pharmaceutical compounds used for the treatment of various cancers, infectious diseases and inflammatory conditions. Many of these can be combined with aptamers and made into “smart nanoparticles” and/or “smart liposomes”. The methods of attaching aptamers to the surface of drug nanoparticles and/or drug liposomes are known to those skilled in the art and a considered within the scope of this invention.

In one embodiment of this invention the aptamer is linked to a polyethylene glycol (PEG) chain which is in turn attached to the surface of the drug nanoparticle or drug liposome. The new pharmaceutical formulation is described as “smart” and “stealthy” in that it can selectively deliver the drug to the disease site and also avoid being destroyed by the liver and the reticuloendothelial system.

Pharmaceuticals that can be used to prepare nanoparticles and liposomes can be broadly classified into the following groups.

The cytotoxic drug group includes the folate inhibitors, pyrimidine analogs, purine analogs, alkylating agents and antibiotics. Specific examples include acivicin, aclarubicin, acodazole, adriamycin, ametantrone, aminoglutethimide, anthramycin, asparaginase, azacitidine, azetepa, bisantrene, bleomycin, busulfan, cactinomycin, calusterone, caracemide, carboplatin, carmustine, carubicin, chlorambucil, cisplatin, cyclophosphamide, cytarabine, dacarbazine, dactinomycin, daunorubicin, dezaguanine, diaziquone, doxorubicin, epipropidine, etoposide, etoprine, floxuridine, fludarabine, fluorouracil, fluorocitabine, hydroxyurea, iproplatin, leuprolide acetate, lomustine, mechlorethamine, megestrol acetate, melengestrol acetate, mercaptopurine, methotrexate, metoprine, mitocromin, mitogillin, mitomycin, mitosper, mitoxantrone, mycophenolic acid, nocodazole, nogalamycin, oxisuran, peliomycin, pentamustine, porfiromycin, prednimustine, procarbazine hydrochloride, puromycin, pyrazofurin, riboprine, semustine, sparsomycin, spirogermanium, spiromustine, spiroplatin, streptozocin, talisomycin, tegafur, teniposide, teroxirone, thiamiprine, thioguanine, tiazofurin, triciribine phosphate, triethylenemelamine, trimetrexate, uracil mustard, uredepa, vinblastine, vincristine, vindesine, vinepidine, vinrosidine, vinzolidine, zinostatin and zorubicin. Also included are the toxins such as ricin and diptheria toxin.

The angiogenesis inhibitors group includes: angiostatin, endostatin and tumstatin.

The antibiotic group includes: penicillin, cephalosporin, griseofulvin, bacitracin, polymyxin B, amphotericin B, erythromycin, neomycin, streptomycin, tetracycline, vancomycin, gentamicin, and rifamycin.

The anti-inflammatory group includes betamethasone, budesonide, cortisone, dexamethasone, hydrocortisone, methylprednisolone, prednisolone, prednisone, and triamcinolone.

The biological response modifier group includes cytokines such as tumor necrosis factor, interferons, angiostatin; immune depressors such as cyclosporine, sirolimus, and triptolide; and immune stimulators such as animal or microbial proteins.

This invention describes the therapeutic advantages of attaching a carrier aptamer to the surface of the drug nanoparticle or drug liposome in order to carry the pharmaceutical compound to the disease site. There are many methods of attaching an aptamer to the surface of the nanoparticle or liposome known to those skilled in the art. In some cases the aptamer is directly linked to the surface of the nanoparticle or liposome. In other cases the aptamer is attached through an intermediate link. For example, the aptamer is chemically linked to a polyethylene glycol (PEG) molecule that is itself bound to the surface of the nanoparticle or liposome.

There are many ways of preparing nanoparticles and liposomes; and there are many ways of attaching aptamers to the surface of these structures. These procedures are known to those skilled in the art and do not affect the novelty of this invention, which is the unique use of aptamers directed at intracellular elements to prepare “smart” nanoparticles and “smart” liposomes to treat cancer and other diseases.

The novelty of this invention to utilize aptamers that are directed against intracellular components that are released into the extracellular environment means that the aptamer:pharmaceutical combinations derived from this invention are not tissue-specific but are in fact necrosis-specific. As many tumors and other diseases have damaged tissue with necrotic areas then these novel pharmaceuticals will have a very broad therapeutic profile and can be used to treat a wide variety of different diseases. For example a particular aptamer can be combined with a cancer drug and used to treat a variety of tumors by causing the drug to localize within the areas of necrosis in the tumor; the same aptamer combined with an antibiotic could be used to treat infectious disease by causing the antibiotic to localize within the areas of necrosis caused by the infective agent; and the same aptamer combined with an immune modulating drug could be used to treat autoimmune disease by causing the drug to localize within the area of inflammation. To illustrate this concept an aptamer that binds to DNA can be used to coat paclitaxel nanoparticles and used to treat tumors; the same anti-DNA aptamer can be used to coat penicillin liposomes and used to treat infections; and the same anti-DNA aptamer can be used to coat cyclosporin liposomes and used to treat autoimmune disease. It is obvious that other aptamers directed against other cellular components can be similarly employed and are within the scope of this invention.

In another embodiment of this invention the same aptamer can be attached to a variety of different drugs and used to treat a particular disease such as cancer: For example, an anti-DNA aptamer can be used to coat paclitaxel nanoparticles and/or methotrexate liposomes and/or doxorubicin liposomes or other cancer drugs and used to treat tumors. It is obvious that other aptamers directed against other cellular components can be similarly employed to treat tumors and are within the scope of this invention.

In another embodiment of this invention the same aptamer can be attached to a variety of antimicrobial drugs and used to treat an infection. For example, an anti-DNA aptamer can be used to coat a variety of different antibiotics formulated into nanoparticles or liposomes. It is obvious that other aptamers directed against other cellular components can be similarly employed to treat infection and are within the scope of this invention.

In another embodiment of this invention the same aptamer can be attached to a variety of anti-inflammatory drugs and used to treat an autoimmune disease. For example, an anti-DNA aptamer can be used to coat a variety of different steroidal and/or immune modulating drugs formulated into nanoparticles or liposomes. It is obvious that other aptamers directed against other cellular components can be similarly employed to treat autoimmune disease and are within the scope of this invention.

Further, in another embodiment of this invention, because there are multiple different intracellular substances that are expressed in necrotic areas multiple different aptamer:drug combinations can be developed to treat a specific disease condition. For example, there would be a list of aptamers manufactured against various intracellular ligands as enumerated earlier. Each of these aptamers can be attached to any one of the pharmaceutical drugs listed earlier to form a novel pharmaceutical. These novel drugs can then be used singly or in combination to treat a wide variety of diseases.

The novelty of this invention in treating cancer is the use of aptamers that are not targeted to tumor associated ligands. For example, the carrier aptamers of this invention that are utilized in treating tumors are not directed against a tumor associated antigen present on viable tumor cells. Instead, the aptamers are directed against normal intracellular components present in both tumor and normal cells that are expressed into the extracellular medium in the areas of necrosis. As such the aptamers have no direct influence upon the tumor cells. However, they are utilized to carry the therapeutic drug to the diseased site within the tumor where the drug can be released and affect the surrounding tumor cells. There are no prior reports of the use of aptamers directed against necrotic material being employed in cancer treatment.

Similarly, the novelty of this invention in treating infectious disease is the use of aptamers that are not targeted to the disease causing organism. Instead, the aptamers are directed against normal intracellular components that are expressed into the extracellular medium in the areas of necrosis. As such the aptamers have no direct influence upon the disease causing organism. However, they are utilized to carry the therapeutic drug to the diseased site within the infected site where the drug can be released and affect the pathogen. There are no prior reports of the use of aptamers directed against necrotic material being employed in the treatment of infectious disease.

Similarly, the novelty of this invention in treating autoimmune disease is the use of aptamers that are not targeted to the systemic immune system. Instead, the aptamers are directed against normal intracellular components that are expressed into the extracellular medium in the areas of inflammation. As such the aptamers have no direct influence upon the inflammatory cells. However, they are utilized to carry the therapeutic drug to the inflamed site where the drug can be released and affect the inflammatory cells within the area of inflammation. There are no prior reports of the use of aptamers directed against necrotic material being employed in the treatment of autoimmune disease.

Depending on the disease to be treated patents will generally receive these drugs parenterally, or by intramuscular injection or by subcutaneous injection, or by direct injection into the disease site. The carrier aptamers are non-immunogenic and patients can therefore receive repeated treatment without developing an allergic reaction to the aptamer-linked drug compounds.

The description and examples presented in this invention are gives as illustration and not as limitation. Those of ordinary skill in the art will recognize from the description and examples given in this invention other embodiments and applications that fall within the spirit and scope of this invention.

What is claimed is:

1. A method of utilizing specific aptamers as carrier agents for drug nanoparticles and/or drug liposomes used in the treatment of cancer and other diseases.

2. A method according to claim 1 whereby the carrier aptamers have the capacity to bind to intracellular components of the cell that are expressed extracellularly within necrotic areas found in tumors, and/or in infectious diseases, and/or in autoimmune diseases and/or at sites of inflammation.

3. A method according to claim 2 whereby the intracellular components being targeted by the carrier aptamer includes nuclear material, microsomal material, mitochondrial material, ribosomal material, the cytoskeleton, and/or other cytoplasmic components that are expressed extracellularly within areas of dead and dying cells.

4. A method whereby the aptamers described in claims 1-2 are used to coat a variety of drug nanoparticles that are manufactured in a variety of ways including solvent precipitation; high pressure homogenization; and other methods known to those skilled in the art.

5. A method whereby the aptamers described in claims 1-2 are used to coat a variety of drug liposomes that are manufactured in a variety of ways including emulsification, and precipitation methods and other methods known to those skilled in the art.

6. A method whereby the aptamers described in claim 1 are used to coat a variety of drug nanoparticles and/or drug liposomes thru a polyethylene glycol molecule link.

7. A method according to claim 4 whereby the drug nanoparticles are prepared from a drug that is insoluble or poorly soluble in physiological solution.

8. A method according to claim 5 whereby the drug liposomes are prepared from a drug that is lipid soluble but insoluble or poorly soluble in physiological solution

9. A method according to claim 5 whereby the drug liposomes are prepared from a drug that is soluble in aqueous or physiological solution.

10. A method according to claim 1 of cancer treatment utilizing a therapeutic dosage of a variety of cytotoxic anti-cancer drugs formulated into aptamer coated nanoparticles or aptamer coated liposomes and injected into the cancer patient.

11. A method according to claim 1 of cancer treatment utilizing a therapeutic dosage of a variety of biological response modifiers formulated into aptamer coated nanoparticles or aptamer coated liposomes and injected into the cancer patient.

12. A method according to claim 1 of cancer treatment utilizing a therapeutic dosage of a variety of toxins formulated into aptamer coated nanoparticles or aptamer coated liposomes and injected into the cancer patient

13. A method according to claim 1 of cancer treatment utilizing a therapeutic dosage of a variety of foreign animal or microbial protein formulated into aptamer coated nanoparticles or aptamer coated liposomes and injected into the cancer patient.

14. A method according to claim 1 of cancer treatment utilizing a therapeutic dosage of a variety of angiogenesis inhibiting compounds formulated into aptamer coated nanoparticles or aptamer coated liposomes and injected into the cancer patient.

15. A method according to claim 1 for treatment of infectious disease utilizing a therapeutic dosage of a variety of antibiotic and anti-microbial drugs formulated into aptamer coated nanoparticles or aptamer coated liposomes and injected into the cancer patient

16. A method according to claim 1 for treatment of autoimmune and inflammatory disease utilizing a therapeutic dosage of a steroidal, or cytotoxic, or immune modulating drug formulated into aptamer coated nanoparticles or aptamer coated liposomes and injected into the cancer patient.
